(19) United States
(12) Patent Application Publication
(10) Pub. No.: US 2017/0209362 A1
(43) Pub. Date: Jul. 27, 2017

(54) METHOD FOR MAKING HERBAL SOAP FOR ALLEVIATING ATOPIC SKIN DISEASE

(71) Applicants: Byung Seung NOH, Gyeongsangnam-do (KR); Se Eung NOH, Jeollabuk-do (KR); Chang Keun KANG, Gyeongsangnamdo (KR)

(72) Inventors: Byung Seung NOH, Gyeongsangnam-do (KR); Se Eung NOH, Jeollabuk-do (KR); Chang Keun KANG, Gyeongsangnamdo (KR)

(21) Appl. No.: 15/003,554

(22) Filed: Jan. 21, 2016

(51) Int. Cl. A61K 8/97; A61K 36/258; A61K 36/25; A61K 36/48; A61K 36/605; A61Q 17/04; A61K 36/70; A61K 36/355; A61K 36/744; A61K 36/21; A61Q 19/10; A61K 8/04; A61K 8/92; A61K 36/484

(52) U.S. Cl. CPC A61K 8/97; A61K 8/925; A61K 36/258; A61K 36/25; A61K 36/48; A61K 36/605; A61K 36/484; A61K 36/70; A61K 36/355; A61K 36/744; A61K 36/21; A61Q 19/10; A61K 8/042; A61Q 17/04; A61K 2800/596; A61K 2800/70

(57) ABSTRACT

Provided is a method for making an herbal soap for alleviating atopic skin disease, the method comprising combining five main medicinal herbs consisting of Kalopanacis cortex, Amaranthus mangostanus, Gardenia jasminoides, Lonicerae flos and Rumex japonicus Houtt with four supplementary medicinal herbs consisting of Glycyrrhiza uralensis Fischer, Mori cortex, Phaseoli radiati semen and red ginseng, which serve to increase the efficacies of the five main medicinal herbs, and refined horse fat containing unsaturated fatty acids and vitamin E, according to an herb combination method that is used in Chinese medicine. According to the method, active ingredients effective for atopic dermatitis patients can be extracted from the medicinal herbs. The herbal extract obtained according to the method exhibits excellent effects on the fundamental treatment of diseases having a high correlation with atopic dermatitis patients and on the alleviation of atopic dermatitis.

### BACKGROUND

The present invention relates to an herbal soap for alleviating atopic skin disease, and more particularly, a method for making an herbal soap for alleviating atopic skin disease, in which the herbal soap contains, as an active ingredient, an herbal extract from a combination of four main medicinal herbs effective for atopic skin disease and four supplementary medicinal herbs serving to increase the efficacies of the five main medicinal herbs, and refined horse fat, and has the effect of fundamentally treating diseases having a high correlation with atopic skin disease and also the effect of alleviating atopic skin disease.

In recent years, soaps have been produced containing herbal medicines which show special functionalities such as prevention of various skin troubles, including atopic skin diseases, and which can reduce skin irritation during hand and face washing and bathing and maintain skin elasticity.

Soap acts as a cleaning agent that separates effete matter or dirt from the body by interfacial surface tension occurring when it comes into contact with water. If secreted sebum is not completely washed out or if horny substance, effete matter, or cosmetic or soap residue clogs skin pores, inflammation will occur, resulting in various skin troubles, including acne.

In addition to the above-described external skin troubles, various skin diseases, including dry skin, dermatomycosis, atopy and the like, are increasing due to allergy caused by environmental fine dust resulting from advanced industrialization, the imbalance of the internal organs of the human body caused by excessive intake of instant foods, an increase in allergenic substances such as house dust mites living in residential spaces (such as artificial wall paper, beds, carpets, sofas, etc.) in apartments made of concrete, abnormal genetic predispositions, abnormal immunological responses, abnormalities in skin barriers, etc.

Skin aging is broadly classified into two types: intrinsic aging that occurs in every people with the passage of time without environmental factors; and extrinsic aging that is caused by environmental factors such as UV rays.

Intrinsic aging is mainly caused by the accumulation of damage to the constituents of the human body, which is caused by reactive oxygen radicals produced in the metabolic processes of the human body, whereas extrinsic aging is caused by inflammatory reactions due to toxic reactive oxygen species and UV rays.

In order to prevent this extrinsic aging, various functional cosmetic products have been actively developed. Such functional cosmetic products are effective in functional terms, but the compositions thereof are mostly chemically synthesized, and oxidized cosmetic compositions can cause skin irritation due to chemical changes. In order to prevent skin aging by removing reactive oxygen species from the skin without skin irritation, studies on cosmetic products comprising natural plant materials have recently been actively conducted.

Korean Patent No. 10-1257641 (entitled “Method for preparing herbal cosmetic composition for skin whitening and wrinkle reduction) owned by the applicant discloses a method of preparing a herbal cosmetic composition of combining three main natural herbal medicines with four supplementary natural medicinal herbs and refined horse fat according to a herbal combination method that is used in Chinese medicine. The three main natural herbal medicines and four supplementary natural herbal medicines used in the above Korean Patent are natural herbal medicines having pharmacological effects on skin whitening and wrinkle reduction.

Thus, the present invention has been made based on the various experiences of the applicant as a professor in the Department of Traditional Chinese Medicine Henan, Henan University of Traditional Chinese Medicine, Henan, China, and as a doctor of traditional Chinese medicine, and the expert knowledge of the applicant, and is intended to provide a novel method for making a herbal soap for alleviating atopic skin disease, in which the herbal soap causes no harm to the skin even upon long-term application and contains, as an active ingredient, extracts obtained by extracting nine natural medicinal herbs selected according to a herb combination method that considers mutual actions between herbs, in which the medicinal herbs grow naturally all over the world and have the effect of fundamentally treating diseases having a high correlation with atopic skin disease and the effect of alleviating atopic skin disease, and refined horse fat.

### SUMMARY OF THE INVENTION

The present invention has been made in order to solve the above-described problems, and it is an object of the present invention to provide a method for making a herbal soap for alleviating atopic skin disease, in which the herbal soap contains, as an active ingredient, an herbal extract obtained by mixing five main medicinal herbs consisting of Kalopanacis cortex, Amaranthus mangostanus, Gardenia jasminoides, Lonicerae flos and Rumex japonicus Houtt with four supplementary medicinal herbs consisting of Glycyrrhiza uralensis Fischer, Mori cortex, Phaseoli radiati semen and red ginseng, which increase the efficacies of the five main medicinal herbs, according to an herb combination method that is used in Chinese medicine, heating and aging the herb mixture to obtain an extract, and adding refined horse fat to the extract, and has the effect of fundamentally treating diseases having a high correlation with atopic skin disease and also the effect of alleviating atopic skin disease.

To achieve the above object, the present invention provides a method for making an herbal soap for alleviating atopic skin disease, the method comprising the steps of:

(1) preparing five main medicinal herbs consisting of Kalopanacis cortex, Amaranthus mangostanus, Gardenia jasminoides, Lonicerae flos and Rumex japonicus Houtt, and four supplementary medicinal herbs consisting of Glycyrrhiza uralensis Fischer, Mori cortex, Phaseoli radiati semen and red ginseng, which serve to increase the efficiencies of the five main medicinal herbs;

(2) introducing Amaranthus mangostanus, Rumex japonicus Houtt and Lonicerae flos, which have excellent effects on the treatment and alleviation of atopic skin disease, among the five main medicinal herbs, into an herb medicine brewing pot together with purified water, and preheating the introduced herbs;

(3) after the preheating in step (2), stopping the heating, and aging the preheated herbs in a state in which the internal pressure of the herb medicine brewing pot was removed, thereby extracting a main raw material for preparing a soap composition;

(4) finely crushing the main medicinal herb Gardenia jasminoides and the supplementary medicinal herbs Glycyrrhiza uralensis Fischer, Mori cortex and Phaseoli radiati semen, prepared in step (1), finely cutting the red ginseng, introducing the crushed herbs and the cut red ginseng into the herb medicine brewing pot together with the main raw material extracted in step (3), and reheating the introduced herbs and main raw material;
(5) after the reheating in step (4), stopping the heating, and then aging the reheated herbs in a state in which the internal pressure of the herb medicine brewing pot was removed;
(6) after the aging in step (5), reheating the content of the herb medicine brewing pot, followed by filtration, thereby recovering a raw material extract for making the soap composition; and
(7) transferring the raw material extract, obtained in step (6), to an agitator, and adding purified horse fat thereto, followed by gelling.

According to an embodiment of the present invention, the five main medicinal herbs prepared in step (1) may be 1,000 g of Kalopanacis cortex, 4,000 g of Amaranthus mangostanus, 1,000 g of Gardenia jasminoides, 1,000 g of Lonicerae flos, and 4,000 g of Rumex japonicus Houtt.

According to another embodiment of the present invention, the four supplementary medicinal herbs prepared in step (1) may be 1,000 g of Glycyrrhiza uralensis Fischer, 1,000 g of Mori cortex, 1,000 of Phaseoli radiati semen, and 500 g of red ginseng.

According to still another embodiment of the present invention, the purified horse fat used in step (7) may be used in an amount of 500 g.

According to still another embodiment of the present invention, the preheating in step (2) may be performed by introducing the medicinal herbs together with 15-20 L of purified water into a 20-30 L herb medicine brewing pot and preheating the introduced medicinal herbs under a pressure of 2-3 kgf/cm² at a temperature of 70° C. to 90° C. for 2-3 hours.

According to still another embodiment of the present invention, the reheating in step (4) may be performed in the herb medicine brewing pot under a pressure of 2-3 kgf/cm² at a temperature of 90° C. to 100° C. for 8-10 hours.

According to still another embodiment of the present invention, the aging in step (3) and the aging in step (5) may each be performed for 10-12 hours.

According to still another embodiment of the present invention, the recovering of the raw material extract in step (6) may be performed by reheating the content of the herb medicine brewing pot at a temperature of 100° C. to 120° C. for 2-3 hours and then filtering the medicinal herbs, contained in the herb medicine brewing pot, through a 200-mesh filter under a pressure of 2-3 kgf/cm².

According to still another embodiment of the present invention, the gelling in step (7) may be performed by heating the raw material extract and the horse fat at a temperature of 90° C. to 100° C. so as not to stick to the agitator, thereby evaporating water.

### BRIEF DESCRIPTION OF THE DRAWINGS

FIG. 1 illustrates the optimal mixing of five main medicinal herbs, four supplementary medicinal herbs and horse fat, which are used in a method for making an herbal soap for alleviating atopic skin disease according to a preferred embodiment of the present invention.

FIG. 2 is a flow chart illustrating a method for making a herbal soap for alleviating atopic skin disease according to an preferred embodiment of the present invention.

FIGS. 3 to 9 are flow charts illustrating methods for making an herbal soap for alleviating atopic skin disease according to various embodiments of the method shown in FIG. 2.

### DETAILED DESCRIPTION OF THE INVENTION

Hereinafter, a method for making an herbal soap for alleviating atopic skin disease according to a preferred embodiment of the present invention will be described in detail with reference to the accompanying drawings.

It should be noted that the same reference numerals are used throughout the different drawings to designate the same or similar components. Further, in the description of the present invention, the detailed description of known functions and configurations incorporated herein will be omitted when it may obscure the subject matter of the present invention.

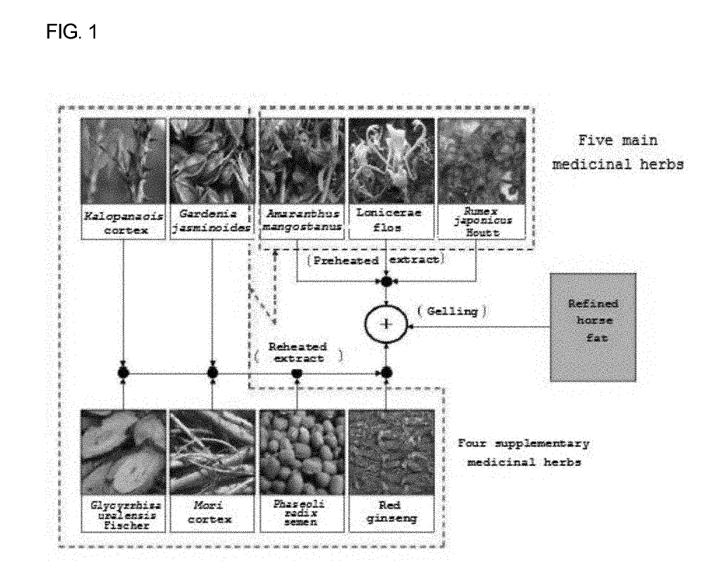

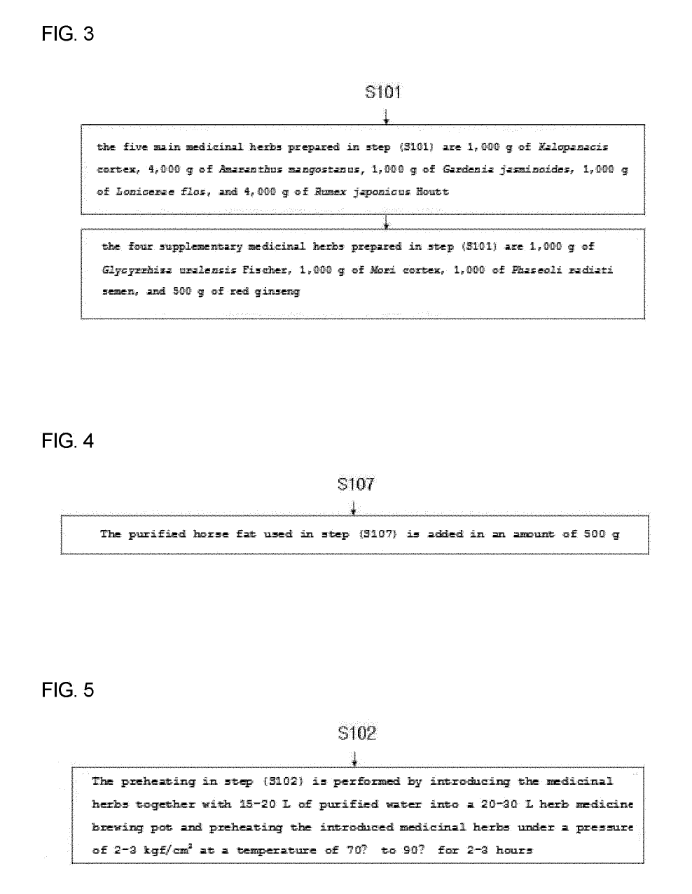

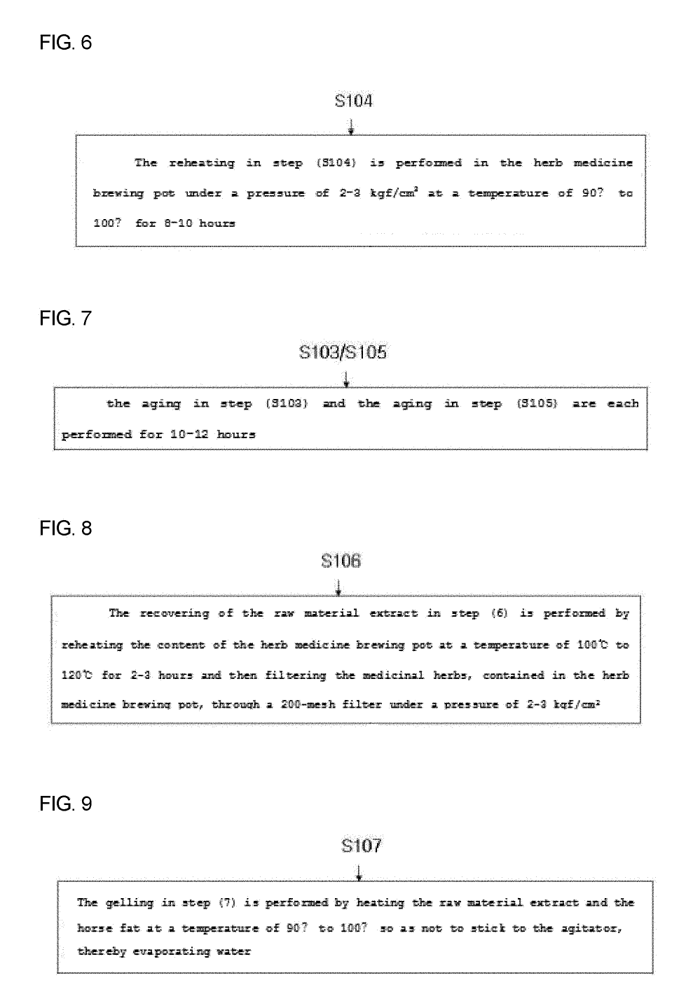

With reference to FIGS. 1 to 9, a detailed description will be made of the characteristics, major components and efficacies of five main medicinal herbs, four supplementary medicinal herbs serving to increase the efficacies of the five main medicinal herbs, and refined horse fat, which are used in methods for making an herbal soap for alleviating atopic skin disease according to embodiments of the present invention.

According to an embodiment of the present invention, the five main medicinal herbs are 1,000 g of Kalopanacis cortex, 4,000 g of Amaranthus mangostanus, 1,000 g of Gardenia jasminoides, 1,000 g of Lonicerae flos, and 4,000 g of Rumex japonicus Houtt, and the four supplementary medicinal herbs serving to increase the efficacies of the five main medicinal herbs are 1,000 g of Glycyrrhiza uralensis Fischer, 1,000 g of Mori cortex, 1,000 of Phaseoli radiati semen, and 500 g of red ginseng.

The five main medicinal herbs (natural medicinal herbs) according to the embodiment of the present invention will now be described with reference to FIG. 1.

(1) Kalopanacis cortex (or Eryhrinae cortex) is used in an amount of 1,000 g in the method for making the soap composition according to the embodiment of the present invention. It has a potent inhibitory effect against skin fungi, and thus exhibits excellent pharmacological effects against skin diseases such as scabies.

More specifically, Kalopanacis cortex is the cortex of Kalopanax pictus Nakai, and is bitter and hot in taste, plain in nature, and is non-toxic. Particularly, it contains alkaloids having an action of inhibiting Staphylococcus aureus and skin fungi. According to Gaebosinsangjeongboncho (a Chinese medical book compiled in the Song dynasty), Kalopanacis cortex is used to treat intestinal convulsions, infections, a long dysentery with bloody excrement and viscous excrement, eliminate skin diseases and scabies, cure pain caused by caries, and also treat skin redness.

(2) Amaranthus mangostanus is used in an amount of 4,000 g in the method for making the soap composition according to the embodiment of the present invention. It acts to eliminate the fat mass cholesterol flowing in human blood vessels, and has an excellent pharmacological effect on the alleviation of atopic dermatitis that appears on the skin.

More specifically, Amaranthus mangostanus refers to the dried whole plant of Partulaca oleracea L., an annual plant belonging to the family Portulacaceae, and is sour in taste and cold in nature, and thus acts on the heart meridian and the large intestine meridian. It acts to lower fever, neutralize toxicity, eliminate blood stasis, kill insects, and facilitate urination. The results of pharmacological studies indicated that Amaranthus mangostanus acts to strengthen the heart, elevate blood pressure, inhibits bacteria, constrict the uterus, and stop bleeding. It is mainly used for the prevention and treatment of colitis. In addition, it is used for boils, gonorrhea, erysipelas, eczema, etc., and is also effective against pulmonary tuberculosis, pulmonary abscess, and arthritis.

(3) Gardenia jasminoides is used in an amount of 1,000 g in the method for making the soap composition according to the embodiment of the present invention. It acts to lower fever in the human body and has antimicrobial activity, and thus exhibits excellent pharmacological effects on the removal of facial discolorations and the alleviation of facial skin diseases such as acne, as well as atopic dermatitis.

More specifically, Gardenia jasminoides refers to the dried fruit of an evergreen shrub belonging to the family Rubiaceae, and is widely used as an antiphlogistic, a diuretic and the like in Chinese medicine. According to Donguibogam (a Korean medicine encyclopedia), Gardenia jasminoides is cold in nature, bitter in taste, and non-toxic, and acts to eliminate fever toxicity in the chest, the large and small intestines and the stomach. Particularly, it is known that when Gardenia jasminoides is used together with Amaranthus mangostanus according to the embodiment of the present invention, it exhibits excellent effects against nephritis, colitis, bloody excrement, hemorrhoids, constipation, leucoderma, dermatitis, facial discolorations, etc. Such effects of Gardenia jasminoides are attributable to the action of flavonoids that are the main components of Gardenia jasminoides.

As used herein, the term “flavonoids” refers to about 4,000 compounds that constitute the yellow color of fruits, vegetables, herbs and the like. The flavonoids are main components attributable to the red, blue and yellow colors of plants, and particularly, are widely distributed in the stem, root bark, pollen and the like of apples (apples, lemons, oranges), vegetables (tomatoes, cabbage, potatoes, onions), and beverage plants (tea, grape wind). In addition, the flavonoids are water-soluble compounds having an important effect on the color and fragrance of these foods. In particular, the flavonoids much distributed in the surface portion of fruits, vegetables and herbs play an important role in protecting these plants from UV rays, and are produced as secondary metabolic products of these plants. These flavonoids are also called vitamin P, and are also found in beans, grains and nuts. Further, these flavonoids are known to exhibit antiviral, antihistamine and antioxidant effects in the body. In addition, these flavonoids act to reduce the risk of development of cancer and assist in the prevention or treatment of various diseases.

(4) Lonicerae flos is used in an amount of 1,000 g in the method for making the soap composition according to the embodiment of the present invention. It acts to eliminate toxins accumulated in the body due to the imbalance of the body's internal organs, and facilitate Qi and blood circulation, and thus has excellent pharmacological effects on the alleviation of atopic dermatitis.

More specifically, Lonicerae flos is the flower of Lonicera japonica, and is white in color upon flowering, which then gradually changes to yellow. Lonicerae flos is cold in nature and sweet in taste, and acts on the blood vessels of the lungs, stomach and heart. The pharmacological effects thereof include antimicrobial effects, anti-inflammatory effects, antipyretic effects, the effect of increasing leukocyte phagocytosis, the effect of exciting the central nervous system, serum cholesterol lowering, ulcer preventing effects, etc. Lonicerae flos contains flavonoids, tannin, alkaloids, saponins, etc. According to B\check{e}nc\check{a}o Gāngmù (a book on Chinese medicine), Lonicerae flos acts to treat infectious diseases, eliminate the pathogenic Qi energy of wind and damp, and cure red swelling and boils. In addition, it acts to treat skin diseases such as syphilis and eliminate Yeolsa (the cause of diseases such as panting, fever, swelling, pain, and constipation) and fever toxin.

Such Lonicerae flos has strong antimicrobial activity and toxicity neutralizing and fever lowering effects, and thus is effective against influenza, flu, infectious inflammation, a boil, etc. In addition, it acts to cure red swelling of the joint, necrotic tissue caused by fever toxicity, Geungoldongtong (the symptom of which has some pain in the bones and sinews), and is also effective against rheumatoid arthritis. Lonicerae flos baked black is also used as a hemostatic for bleeding caused by injury, and Lonicerae flos also has the effect of relieving inflammations such as mastitis or endometritis.

In particular, Lonicerae flos is widely used as therapeutic agents in combination with other medicinal herbs, and is used to treat seborrheic hair loss or seborrheic dermatitis, which is caused by the severe imbalance of the body's internal organs due to the accumulation of mental fatigue and stress. Further, it acts to eliminate toxins from the body and facilitate Qi and blood circulation, and thus is also used to treat atopic dermatitis and acne.

(5) Rumex japonicus Houtt is used in an amount of 4,000 g in the method for making the soap composition according to the embodiment of the present invention. Pharmacologically, it acts to prevent various skin diseases, skin allergy and skin troubles, and prevent the skin from being aged rapidly, thereby whitening the skin and reducing skin wrinkles.

More specifically, Rumex japonicus Houtt a perennial herb plant belonging to the family Polygonaceae, is widely distributed in a little damp places, such as sunny field, farmland, vacant lot, etc. Pharmacologically, it acts to fundamentally cure renal and hepatic diseases having a high correlation with skin diseases, and thus is especially effective in treating skin diseases such as swells or boils. Further, the long-term administration of Rumex japonicas Houtt is excellent in effects to make intestines and blood clean and skin texture smooth, eliminate inflammation and kill various germs, so it can be used in the treatment of all inflammatory diseases and cancers, such as gastritis, stomach ulcer, stomach cancer, etc. In addition, it is also effective in relieving fever and promoting induction of perspiration, consequently with a therapeutic effect for cold, pulmonary tuberculosis, cough, and bronchitis, and remarkably useful to treat a kidney infection or difficult urination that has a direct influence on the hair loss.

As described above, the five main medicinal herbs that are in the method for making the soap composition according to the embodiment of the present invention are different in nature from one another, but have little or no toxicity and exhibit excellent pharmacological effects against skin diseases and are also used as natural medicinal herbs for alleviating skin diseases.

Thus, in the embodiment of the present invention, a method for making a soap composition effective for atopic dermatitis patients with fungal skin, swells or the like is provided which is based on an herb combination method that provides synergism between medicinal herbs having similar efficacies and pharmacological effects so as to increase the original effects (treatment and alleviation of skin diseases), when combined according to the rules of seven emotions and herbal combination.

Hereinafter, a detailed description will be made of the four supplementary medicinal herbs serving to increase the efficacies of the five main medicinal herbs according to the embodiment of the present invention.

(6) Glycyrrhiza uralensis Fischer is used in an amount of 1,000 g in the method for making the soap composition according to the embodiment of the present invention. It acts to neutralize the actions of the other medicinal herbs, does perform hepatic detoxification to restore the functions of the liver. Especially, it contains components liquidritin, liquiritigenin, etc., which inhibit the occurrence of digestive ulcer, and promotes collagen synthesis and hyarulonnic acid synthesis, and inhibit melanin production. Owing to these effects, it is effective in alleviating skin diseases.

More specifically, Glycyrrhiza uralensis Fischer is a perennial plant belonging to the family Fabaceae, and the principal components thereof include glysyrihizic acid, glabridin, saponaretin, licuracid as a kind of flavonoid, flavonoids, tripene-based saponins, etc. Regarding the efficacies, Glycyrrhiza uralensis Fischer has tranquil nature, neutralizes the toxicity of all drugs, gives a sweet flavor without toxicity and combines with a toxic substance to provide a detoxification function, thereby restoring the functions of the liver and exerting a good therapeutic effect in the treatment of drug addiction, infection, hives, dermatitis, eczema, etc.

In particular, among the principal ingredients of Glycyrrhiza uralensis Fischer, the glysyrihizic acid, collected from the root of Glycyrrhiza uralensis Fischer, is 30 to 50 times as sweet as sugar, making it possible to use Glycyrrhiza uralensis Fischer to prepare an artificial sweetener, and is effective in inhibiting occurrence of cancer and tumor, reducing the cholesterol level to improve hardening of the arteries, and protecting the liver. In addition, the glabridin has an antioxidant effect and an inhibitory effect on the production of melanin pigments, and the flavonoids have an anticancer function due to its strong antioxidant effect.

(7) Mori cortex is used in an amount of 1,000 g in the method for making the soap composition according to the embodiment of the present invention. It has excellent pharmacological effects, including antiviral and anti-allergic effects.

More specifically, Mori cortex is the root bark of mulberry trees on which silkworms grow. It is cold in nature, and thus acts as a cough remedy to lower fever in the lungs, and also has excellent antiviral and anti-allergic effects. Further, it promotes the proliferation of lymphocytes to activate the immune system, and activates anti-inflammatory responses. In addition, Mori cortex acts to facilitate urination and promote blood pressure lowering, and thus is effective for edema patients and hypertension patients. Mori cortex has little or no side effects, unlike conventional antidepressant drugs, but is cold in nature, and for this reason, is preferably administered in small amounts to persons having a cold constitution.

(8) Phaseoli radiati semen is used in an amount of 1,000 g in the method for making the soap composition according to the embodiment of the present invention. Pharmacologically, it acts to remove effete matter from the body, lower fever, stimulate appetite, and treat ascites and various skin diseases such as heat rashes, acne and boils.

More specifically, Phaseoli radiati semen, an annual plant belonging to the family Fabaceae, is cold in nature and sweet in taste, is not toxic, and has no side effects. Phaseoli radiati semen contains vitamins, proteins, carbohydrates, and essential oils, which alleviate Baekdok (the nature of a disease of falling out the hair with developing dandruff on the head), strengthen vigor of the body, and balance the five viscera. In addition, it lowers fever in the body, facilitates urination, and promotes the twelve meridians of the body.

In Chinese medicine clinical trials, Phaseoli radiati semen extracts have been used as herbal medicines for neutralizing toxicity, and have been proven to be effective in treating dermatitis, and thus are currently used as agents for preventing makeup allergy or acne in medical drugs and cosmetics.

(9) Red ginseng is used in an amount of 500 g in the method for making the soap composition according to the embodiment of the present invention. It pharmacologically acts to enhance the immunity of the human body, promote fatigue recovery, exert an antioxidant function, remove environmental hormones, remove stress, and improve cardiac and hepatic functions.

More specifically, red ginseng, a kind of Chinese medicine, is the steamed and dried root of Panax ginseng C. A. Meyer (belonging to the family Araliaceae). In particular, because neutralizing activity occurs during the preparation of red ginseng, red ginseng has very excellent absorption ability and does not cause any problem even when it is administered to persons having high fever. Red ginseng contains saponin as a major active ingredient.

It contains not less than 0.10% of ginsenoside Rg1 (C₄₂H₇₂O₁₄: 8 01.01) and not less than 0.20% of of ginsenoside Rb1 (C₅₄H₉₂O₂₃: 1109.29), calculated on the basis of drie d material. It inhibits collagen degradation in vivo and promotes collagen synthesis to thereby prevent aging. In addition, it reinforces calcium in vivo and promotes brain activity, thereby promoting fatigue recovery.

Meanwhile, because the herbal soap for alleviating atopic skin disease according to the embodiment of the present invention is applied to the skin of atopic patients, it contains refined horse fat which is widely used as a cosmetic raw material.

Horse fat is used in an amount of 500 g in the method for making the soap composition according to the embodiment of the present invention. It is rich in unsaturated fats and vitamin E (wheat germ oil), which is what makes the skin high in absorbability and water retention capacity, to effectively treat atopic skin disease and prevent the soap product of the embodiment of the present invention from being decomposed.

The horse fat is a fat extracted from the adipose tissue of a horse and contains, as principal ingredients, about 40% saturated fat and 60% unsaturated fat, which is made up of 7.3% of palmitoleic acid, 34.8% of oleic acid, 14.2% linoleic acid, 0.9% linolenic acid, etc. This makes the horse fat closer to a vegetable oil rather than a fat from animals.

The unsaturated fatty acids contained in the horse fat have efficacies to form cell membranes in the body, eliminate cholesterol, provides better use feelings on the skin than saturated fatty acids, and exhibits high levels of absorbability and water retention capacity to offer a skin protection function, a cell regeneration promoting function, a UV-screening function, and an antibacterial function.

The reason that the horse fat has such efficacies lies in the fact that the horse fact can penetrate deep into the skin pores. As the human skin has minutely uneven surface as well as pores that keep a tiny amount of air remaining in the subcutaneous tissue, the horse fat applied to the skin can penetrate deep into the skin to the extent that even the air remaining in the pores becomes expelled from the pores.

Further, the horse fat that penetrates into the subcutaneous tissue to the depth of 1 mm cannot just capture the germs from the outside through a wound such as a cut, etc. within the skin oil to bring the activity of cells to a stop but also inhibit the invasion of external germs into the skin, thereby with functions to prevent bacterial infection and cure a wound.

In addition, the horse fat is much similar in the compositional ingredients to the human fat, so it can penetrate into the subcutaneous tissue and become a nutrient, which is a human-friendly substance having no harm to the skin when absorbed into the blood, thereby promoting blood circulation and accelerating metabolisms.

### Embodiment

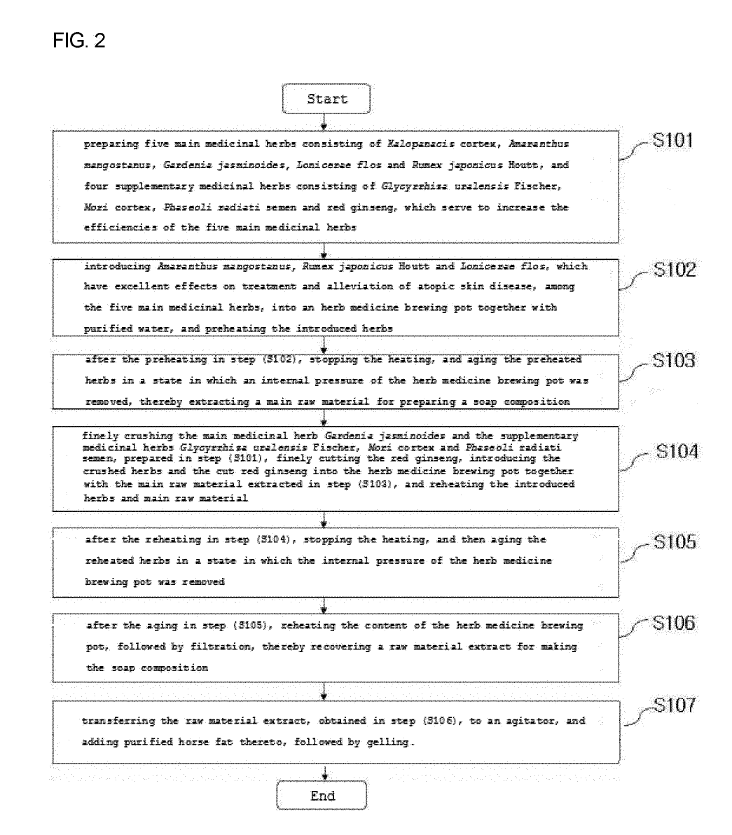

Hereinafter, a method for making an herbal soap for alleviating atopic skin disease according to an embodiment of the present invention will be described in detail with reference to FIGS. 2 to 9.

First, a method according to an embodiment of the present invention comprises step (1) (S101) of preparing five main medicinal herbs consisting of Kalopanacis cortex, Amaranthus mangostanus, Gardenia jasminoides, Lonicerae flos and Rumex japonicus Houtt, and four supplementary medicinal herbs consisting of Glycyrrhiza uralensis Fischer, Mori cortex, Phaseoli radix semen and red ginseng, which serve to increase the efficiencies of the five main medicinal herbs.

The five main medicinal herbs that are used in the embodiment of the present invention are 1,000 g of Kalopanacis cortex, 4,000 g of Amaranthus mangostanus, 1,000 g of Gardenia jasminoides, 1,000 g of Lonicerae flos, and 4,000 g of Rumex japonicus Houtt.

In addition, the four supplementary medicinal herbs that are used in the embodiment of the present invention are 1,000 g of Glycyrrhiza uralensis Fischer, 1,000 g of Mori cortex, 1,000 of Phaseoli radiati semen, and 500 g of red ginseng.

The reason why the five main medicinal herbs and the four supplementary medicinal herbs are combined with one another according to the embodiment of the present invention are that these are natural medicinal herbs for treating and alleviating various skin diseases. In particular, the five main medicinal herbs and the four supplementary medicinal herbs serving to increase the efficacies of the five main medicinal herbs, which are used in the embodiment of the present invention, are natural medicinal herbs selected according to the rules of seven emotions and herbal combination, which are herbal combination methods that allow side effects to be somewhat offset between medicinal herbs. Thus, although there is a slight difference in nature between these selected medicinal herbs, these medicinal herbs are all not-toxic and show synergistic effects, and thus can exhibit excellent pharmacological effects in atopic dermatitis patients, and can be applied even to not only persons having a weak constitution, but also Soyangin or Soeumin, without causing side effects.

In other words, the embodiment of the present invention is characterized in that, when the five main medicinal herbs having partially similar effects against atopic skin disease are combined with the four supplementary medicinal herbs, an herb combination method that provides mutual enhancement between herbs is used, which allows the four supplementary medicinal herbs to increase the effects of the five main medicinal herbs on the treatment and alleviation of atopic skin disease.

Step (1) (S101) is followed by step (S102) of introducing Amaranthus mangostanus, Rumex japonicus Houtt and Lonicerae flos, which have excellent effects on the treatment and alleviation of atopic skin disease, among the five main medicinal herbs, into an herb medicine brewing pot together with purified water, and preheating the introduced herbs.

Herein, the specific reason why Amaranthus mangostanus, Rumex japonicus Houtt and Lonicerae flos are heated in the embodiment of the present invention is because Amaranthus mangostanus (that is an annual plant belonging to the family Portulacaceae), Rumex japonicus Houtt (that is a perennial plant belonging to the family Polygonaceae) and Lonicerae flos (that is the flower of Lonicera japonica) are not the hard stems or roots of tree branches, but are the natural herbs and flower petals of soft portions, and for this reason, these medicinal herbs can be changed due to high heat before their extraction when these herbs are heated at high temperatures in the initial stage. Thus, the reason is to prevent a change in the medicinal herbs and to enable the medicinal herbs to be extracted slowly at low temperatures so that the components of the medicinal herbs, which are effective for treating and alleviating atopic skin disease, can be sufficiently extracted.

In other words, Amaranthus mangostanus, Rumex japonicus Houtt and Lonicerae flos are the natural medicinal herbs critical in the method for making the herbal soap for alleviating atopic skin disease according to the embodiment of the present invention. These natural medicinal herbs are separately preheated to extract a main raw material for preparing the soap composition. These natural medicinal herbs may be used after drying, but when raw medicinal herbs are used without drying, desired medicinal components can be extracted in increased amounts. Thus, in the embodiment of the present invention, raw medicinal herbs are washed clean, preheated and aged to extract a main raw material having excellent effects on the alleviation of atopic skin disease. The extract is combined again with the other main medicinal herbs and the four supplementary medicinal herbs, and then reheated and further aged to extract a raw material for making the soap composition, which has increased pharmacological effects.

In addition, the reason why Amaranthus mangostanus is used as one of the five main medicinal herbs is as follows. An event caused by the resistance of the skin tissue and cell tissue of the human body to external viruses is referred to as inflammations, and inflammations include an inflammation occurring in the body, and dermatitis appearing on the skin surface. It is known that Amaranthus mangostanus showing an excellent effect against dermatitis appearing on the skin surface is very effective against atopic patients. In addition, if fat accumulates in the human body, it can cause various vascular diseases and obesity. Fat flowing in blood vessels is called cholesterol, which is not dissolved and adheres to the blood vessel wall or clogs blood vessels. Amaranthus mangostanus is known to have a very excellent effect on the removal of this cholesterol. In addition, it is known that Amaranthus mangostanus activates brain function and has a very excellent effect on the prevention of dementia that occurs as brain neurons and cells decrease with age. The reason is because Amaranthus mangostanus contains a large amount of omega-3 that prevents human aging to enhance brain function and maintain the brain healthy.

In the embodiment of the present invention, the preheating in step (2) is performed by introducing the medicinal herbs together with 15-20 L of purified water into a 20-30 L herb medicine brewing pot and heating the introduced medicinal herbs under a pressure of 2-3 kgf/cm² at a temperature of 70 to 90° C. for 2-3 hours.

Herein, the capacity of the brewing pot, the amount of the purified water, the preheating pressure, temperature and time in the embodiment of the present invention are not simple values, but are important facts. This is because the natural medicinal herbs are combined according to the properties and pharmacological effects thereof using the rule of herbal combination that considers mutual actions between herbs, and the capacity of the brewing pot, the amount of purified water, and the preheating temperature, pressure and time are previously calculated and determined according to the combination in order to obtain the desired raw material extract depending on the intended use.

In other words, a herb medicine brewing pot unsuitable the weight (g) of the combined herbs cannot be used, and if the ratio between the drugs and purified water introduced into the herb medicine brewing pot is not proper, the desired extract cannot be produced, and if the heating temperature, pressure and time are not accurate, the herbs will be burned or the raw material extract cannot be obtained. Thus, in the embodiment of the present invention, the above values are critical values determined according to the properties and pharmacological effects of the five main medicinal herbs and the four supplementary medicinal herbs using the rule of herbal combination.

After the preheating in step (2), step (3) is performed, in which the heating is stopped, followed by aging in a state in which the internal pressure of the herb medicine brewing pot was removed, thereby extracting a raw material for making the soap composition.

The specific reason why step (3) according to the embodiment of the present invention is performed is because Amaranthus mangostanus (that is an annual plant belonging to the family Portulacaceae), Rumex japonicus Houtt (that is a perennial plant belonging to the family Polygonaceae) and Lonicerae flos (that is the flower of Lonicera japonica) are natural raw medicinal herbs having excellent pharmacological effects on atopic dermatitis patients, and for this reason, these medicinal herbs are easily mixed with one another with slow cooling, so that they have maximized pharmacological effects when they are mixed with the remaining main medicinal herbs and the four supplementary medicinal herbs in step (4).

On other words, when the main raw materials extracted from the herbs are mixed while they are cooled slowly, an extract from Amaranthus mangostanus can exhibit excellent pharmacological effects of removing the fat mass cholesterol flowing in human blood vessels and alleviating atopic dermatitis; an extract from Rumex japonicus Houtt can exhibit pharmacological effects of preventing various skin diseases, skin allergy and skin troubles and preventing rapid aging of the skin to whiten the skin and reduce skin wrinkles; and an extract from Lonicerae flos can exhibit excellent pharmacological effects of releasing toxins accumulated in the body by the imbalance of body's internal organs caused by stress. Thus, these herbs are natural raw medicinal herbs having similar pharmacological effects. Accordingly, the embodiment of the present invention is characterized in that these herbs having similar pharmacological effects are combined according to an herbal combination method that provides synergism between the herbal medicines, so that their effects on the treatment and alleviation of atopic dermatitis can be maximized through the synergism thereof.

Particularly, in the embodiment of the present invention, the effects of the soap composition on the treatment and alleviation of atopic dermatitis are maximized by adding Lonicerae flos that is used to treat seborrheic hair loss or seborrheic dermatitis and release toxins accumulated in the body and also facilitate Qi and blood circulation to thereby treat atopic dermatitis and acne.

Step (3) is followed by step (4) (S104) of finely crushing the main medicinal herb Gardenia jasminoides and the supplementary medicinal herbs Glycyrrhiza uralensis Fischer, Mori cortex and Phaseoli radiati semen, prepared in step (1), finely cutting the red ginseng, introducing the crushed herbs and the cut red ginseng into the herb medicine brewing pot together with the raw material extracted in step (3), and reheating the introduced herbs and raw material.

The specific reason why the reheating in step (4) is performed is because Kalopanacis cortex (that is the cortex of Kalopanax pictus Nakai), Gardenia jasminoides (that is the dried fruit of an evergreen shrub belonging to the family Rubiaceae), Glycyrrhiza uralensis Fischer (that is a perennial plant belonging to the family Fabaceae), Phaseoli radiati semen (that is the fruit of an annual plant belonging to the family Fabaceae), and red ginseng (that is the steamed and dried root of ginseng) are all stiff and hard medicinal herbs, and for this reason, the medicinal components of the herbs for treating and alleviating atopic skin disease can be sufficiently extracted by heating these herbs at high temperature and pressure.

Particularly, among the five main medicinal herbs that are used in the embodiment of the present invention, Kalopanacis cortex has a potent inhibitory effect against skin fungi, and thus an excellent pharmacological effect against skin diseases such as scabies.

In addition, among the five main medicinal herbs, Gardenia jasminoides acts to lower fever in the human body and exhibits antimicrobial activity, and thus has excellent pharmacological effects on the removal of facial discolorations, facial skin diseases such as acne, and atopic dermatitis.

Among the four supplementary medicinal herbs, Glycyrrhiza uralensis Fischer acts to neutralize the activities of the other medicinal herbs, and binds and removes toxic substances in the liver, thereby restoring the functions of the liver and exerting a good therapeutic effect in the treatment of drug addiction, infection, hives, dermatitis, eczema, etc.

In addition, among the four supplementary medicinal herbs, Mori cortex is the root bark of mulberry trees on which silkworms grow, and exhibits excellent pharmacological effects, including antiviral and anti-allergic effects.

Furthermore, among the four supplementary medicinal herbs, Phaseoli radiati semen pharmacologically acts to remove effete matter from the body, lower fever, stimulate appetite, and treat ascites and various skin diseases such as heat rashes, acne and boils.

Among the four supplementary medicinal herbs, red ginseng pharmacologically acts to enhance the immunity of the human body, promote fatigue recovery, exert an antioxidant function, remove environmental hormones, remove stress, and improve cardiac and hepatic functions.

As described above, Kalopanacis cortex that is used as one of the main medicinal herbs in the embodiment of the present invention has a potent inhibitory effect against skin fungi, and Gardenia jasminoides exhibits antimicrobial activity to thereby remove facial discolorations and alleviate facial skin diseases and atopic skin dermatitis. In addition, Glycyrrhiza uralensis Fischer among the supplementary medicinal herbs binds and removes toxic substances in the liver to thereby alleviate skin diseases, and Mori cortex exhibits excellent pharmacological effects, including antiviral and anti-allergic effects, and Phaseoli radiati semen pharmacologically acts to remove effete matter accumulated in the human body to thereby treat skin diseases. In addition, red ginseng pharmacologically acts to improve liver function, enhance the immunity of the human body, and promote fatigue recovery, thereby alleviating skin diseases.

Particularly, in the embodiment of the present invention, Glycyrrhiza uralensis Fischer and red ginseng are used as the supplementary medicinal herbs according to an herbal combination method that provides synergism between two or more herbal medicines having similar efficacies, thereby doubling the original efficacies thereof through synergism.

In addition, in order to relieve the cold nature of Mori cortex among the supplementary medicinal herbs, red ginseng is used together with Mori cortex according to an herb combination method for mutual antagonism, which allows one drug to eliminate or reduce the efficacy of another drug.

Furthermore, red ginseng among the supplementary medicinal herbs can cause side effects, including hypertension, skin rash, and diarrhea, when it is administered to persons having high fever. For this reason, according to an herb combination method for mutual inhibition which allows the toxicity and or side effects of one drug to be reduced by another drug, and an herb combination method for mutual detoxication which allows one drug to eliminate the toxicity and side effects of another drug, Phaseoli radiati semen together with red ginseng is used as a supplementary medicinal drug so as to somewhat offset fever caused by red ginseng. Herein, the use of Phaseoli radiati semen does not reduce the efficacy of red ginseng.

The functions of red ginseng to enhance the immunity of the human body and promote fatigue recovery, and the pharmacological effects of Phaseoli radiati semen on enhancement of human immunity, fatigue recovery, antioxidant activity, removal of environmental hormones, stress relief, improvement in cardiac and hepatic functions, etc., act to enhance the skin disease treatment and alleviation effects of the five main medicinal herbs.

In addition, Glycyrrhiza uralensis Fischer acting to neutralize the activities of the other medicinal herbs is added, thereby preventing toxicity from occurring in step (4).

Furthermore, the reheating in step (4) is performed in the herb medicine brewing pot under a pressure of 2-3 kgf/cm² at a temperature of 90° C. to 100° C. for 8-10 hours.

Herein, the values used in the reheating in step (4) are critical values determined according to the natures and pharmacological effects of the five main medicinal herbs and the four supplementary medicinal herbs using the rule of herbal combination, as described above with respect to the preheating in step (2).

After the reheating in step (4), step (5) (S105) is performed, in which the heating is stopped, followed by aging in a state in which the internal pressure of the herb medicine brewing pot was removed.

The specific reason why the aging in step (5) is performed is so that the medicinal properties of the different medicinal herbs extracted in steps (2) to (4) are naturally combined through aging and mixing while the medicinal herbs are cooled slowly.

This embodiment of the present invention is based on the herb combination method that provides mutual enhancement between the main and supplementary medicinal herbs having partially similar efficacies as provided in the rules of seven emotions and herb combination, so as to allow the main medicinal herbs to increase the original efficacies of the main medicinal herbs.

In other words, the herb combination method is a method in which two or more herbal medicines selected depending on the state of disease, the performance and efficacy of the medicines and the method for application of the medicines are combined with one another, and is a major form in the application of herbal medicines. This herb combination method is a unique characteristic in the method for making the soap composition for treating and alleviating atopic skin disease according to the present invention.

In the embodiment of the present invention, the aging in step (3) and the aging in step (5) are performed for 10-12 hours.

Herein, the aging time in step (5) is a critical value determined according to the properties and pharmacological efficacies of the five main medicinal herbs and the four supplementary medicinal herbs on the basis of the herb combination rule, as described above for the preheating method in step (2).

After the aging in step (5), step (6) (S106) is performed in which the content in the herb medicine brewing pot is reheated, and then filtered, thereby recovering a raw material extract for making the soap composition.

In the embodiment of the present invention, the recovering of the raw material extract in step (6) is performed by reheating the content of the herb medicine brewing pot at a temperature of 100 to 120° C. for 2-3 hours, and the filtering the content of the herb medicine brewing pot through a 200-mesh filter under a pressure of 2-3 kgf/cm².

The specific reason why the filtration is performed is because the five main medicinal herbs and four supplementary medicinal herbs that are used in the embodiment of the present invention are composed of the root and stem of trees and the leaf and petal of plant, and for this reason, impurities are produced in the heating process and need to be removed by filtration through a 200-mesh filter. After the aging in step (5), the reheating in the herb medicine brewing pot at a high temperature of 100-120° C. is performed in order to remove toxicity resulting from incompatible combination that can cause toxicity and side effects when combining one drug with another drug.

Finally, step (7) (S107) is performed in which the raw material extract recovered in step (6) is transferred to an agitator and refined horse fat is added thereto, followed by gelling.

The refined horse fat that is used in the embodiment of the present invention is added in an amount of 500 g, and the gelling is performed by reheating the herbal extract and the refined horse fat at a temperature of 90-100° C. so as not to stick to the agitator, thereby evaporating water.

The soap composition prepared by the method for making a herbal soap for alleviating atopic skin disease according to the preferred embodiment of the present invention was tested for its pH and toxicity by the Korea Testing & Research Institute (KTR), and the results of the test are shown in Table 1 below.

TABLE 1
Toxicity Test for Soap Composition for Alleviating Atopic Skin Disease
Sample name: soap composition for alleviating atopic skin disease
Test results

| | | Sample | | |
|---|---|---|---|---|
| Test items | Unit | classification | Results | Test method |
| pH | μg/g | — | 5.1 | Notification No. 2013-24 of the Korean |
| | | | | Ministry of Food and Drug Safety |
| Lead | μg/g | — | Not detached | Notification No. 2013-24 of the Korean |
| | | | | Ministry of Food and Drug Safety |
| Arsenic | μg/g | — | Not detached | Notification No. 2013-24 of the Korean |
| | | | | Ministry of Food and Drug Safety |
| Mercury | μg/g | — | Not detached | Notification No. 2013-24 of the Korean |
| | | | | Ministry of Food and Drug Safety |
| Methanol | v/v % | — | Not detached | Notification No. 2013-24 of the Korean |
| | | | | Ministry of Food and Drug Safety |
| DEHP | μg/g | — | Not detached | Notification No. 2013-24 of the Korean |
| | | | | Ministry of Food and Drug Safety |
| DBP | μg/g | — | Not detached | Notification No. 2013-24 of the Korean |
| | | | | Ministry of Food and Drug Safety |
| BBP | μg/g | — | Not detached | Notification No. 2013-24 of the Korean |
| | | | | Ministry of Food and Drug Safety |
| Methyl paraben | mg/kg | — | Not detached | Guideline for Analytical Method of |
| | | | | Limited Ingredients in Cosmetics (2010) |
| Ethyl paraben | mg/kg | — | Not detached | Guideline for Analytical Method of |
| | | | | Limited Ingredients in Cosmetics (2010) |
| Propyl paraben | mg/kg | — | Not detached | Guideline for Analytical Method of |
| | | | | Limited Ingredients in Cosmetics (2010) |

—Method Detection Limit (MDL)—
Pb: 2 μg/g
As: 1 μg/g
Hg: 0.05 μg/g
Methanol: 0.025 (v/v %)
Dibutylphthalate (DBP): 5 μg/g
Benzylbutylphthalate (BBP): 5 μg/g
Bis(2-ethylhexyl)phthalate (DEHP): 5 μg/g
Methyl paraben: 10 mg/kg
Ethyl paraben: 10 mg/kg
Propyl paraben: 10 mg/kg

As can be seen from the results in Table 1 above, the herbal extract-based soap composition (gelled soap sample) for alleviating atopic skin disease according to the embodiment of the present invention, which comprises the five main medicinal herbs, the four supplementary medicinal herbs serving to increase the efficacies of the five main medicinal herbs, and the refined horse fat rich in unsaturated fatty acids and vitamin E, had a pH of 5.1 and was nontoxic, and no toxic component was detected, indicating that the soap composition is harmless to the human body. This is because the five main medicinal herbs consisting of Kalopanacis cortex, Amaranthus mangostanus, Gardenia jasminoides, Lonicerae flos and Rumex japonicus Houtt, and the four supplementary medicinal herbs consisting of Glycyrrhiza uralensis Fischer, Mori cortex, Phaseoli radiati semen and red ginseng, which serve to increase the efficacies of the five main medicinal herbs, are all natural medicinal herbs collected from nature, and because the composition contains the horse fat closer to vegetable oil than to animal oil.

Skin safety tests for the herbal extract-based soap composition (gelled soap sample) prepared by the method for making a herbal soap for alleviating atopic skin disease according to the preferred embodiment of the present invention were carried out as follows.

### Test Example 1: Skin Irritability Test

In order to examine the skin stability of the soap composition according to the embodiment of the present on 20 adult men and women and 20 atopic dermatitis patients, a 3×6 cm patch (the herbal extract prepared by the method for making a herbal soap for alleviating atopic skin disease according to the embodiment of the present invention) was applied to the forearm of each of the test subjects, and after 48 hours, the applied forearm was observed to determine whether erythema and edema occurred.

The patch test reactions were scored according to the guidelines of the International Contact Dermatitis Research Group (ICDRG), and the mean response rate was calculated using the following equation 1. Criteria and scores for evaluating the patch test reactions are shown in Table 2 below, and the results of calculating the mean response rate are shown in Table 3 below.

TABLE 2
Criteria and Scores for Evaluation of Patch Test Reactions

| Symbol | Score | Criteria for evaluation |
|---|---|---|
| − | 0 | No reaction |
| ± | 0.5 | Faint erythema or slight erythema |
| + | 1 | Well-defined but mild erythema, edema and papule |
| ++ | 2 | Distinct erythema, papule and vesicles |
| +++ | 3 | Severe erythema and bulla, and crust formation |

Mean response rate (%)=(score×number of reactions×100×½)/[3(maximum score)×total number of subjects(n)] Equation 1

TABLE 3
Evaluation Results for Patch Test
Reactions and Mean Response Rate

| | After 48 hours | | | | Response rate |
|---|---|---|---|---|---|
| Test material | ± | + | ++ | +++ | (n = 20) |
| Embodiment | − | − | − | − | 0.00 |

As can be seen in Tables 1 and 2 above, the skin stability of the herbal extract-based soap composition prepared by the method for making a herbal soap for alleviating atopic skin disease according to the embodiment of the present invention was tested, and as a result, it was found that, because the five main medicinal herbs and the four supplementary medicinal herbs serving to increase the efficacies of the five main medicinal herbs are all nontoxic natural medicinal herbs collected from nature and the refined horse fat is a human-friendly material, the soap composition is a safe soap composition causing no skin irritation.

### Test Example 2: Test for Effect on Atopic Dermatitis

In order to examine whether the herbal extract-based soap composition prepared by the method for making an herbal soap for alleviating atopic skin disease according to the embodiment of the present invention is effective against atopic dermatitis, test subjects (guardians in the case of babies) to use the soap composition twice (morning and evening) a day for three months, and then the degree of alleviation of atopic dermatitis was examined by questionnaire and visual observation. The results of the examination are shown in Table 4 below.

TABLE 4
Test for Effect on Atopic Dermatitis

| | | Test results | | | | |
|---|---|---|---|---|---|---|
| Age of subjects | Number of subjects | ++ | + | ± | − | Alleviation rate (%) |
| 0-1 years old | 15 | 8 | 4 | 2 | 0 | 80.00 |
| 1-5 years old | 15 | 10 | 3 | 2 | 0 | 86.66 |
| 5-10 years old | 15 | 9 | 2 | 3 | 1 | 73.33 |
| 10-15 years old | 15 | 10 | 3 | 1 | 1 | 86.66 |
| 15-20 years old | 15 | 8 | 2 | 3 | 2 | 66.66 |
| 20-30 years old | 15 | 9 | 4 | 1 | 1 | 86.66 |
| Over 30 years old | 15 | 10 | 2 | 3 | 0 | 80.00 |
| Total | 105 | 64 | 20 | 15 | 5 | 80.00 |

Criteria for judgment
++: very good;
+: good;
±: moderate;
−: not effective.

Alleviation rate (%)=[number of (++) plus number of (+)]×100/number of subjects Equation 2

As can be seen in Table 4 above, the atopic dermatitis-alleviating effect of the herbal extract-based soap composition prepared by the method for making the herbal soap for alleviating atopic dermatitis according to the embodiment of the present invention was tested, and as a result, the soap composition showed an effect on the alleviation of atopic dermatitis in 84 (80.00%) of 105 test subjects with atopic dermatitis.

In the test results, the soap composition according to the embodiment of the present invention showed an atopic dermatitis-alleviating effect in 73.3% of 5-10 years old children and 66.66% of 15-20 years old youth, and this was believed to be because these results were associated with high activity, unlike those for the other age groups.

Thus, it was found that, when the five main medicinal herbs consisting of Kalopanacis cortex, Amaranthus mangostanus, Gardenia jasminoides, Lonicerae flos and Rumex japonicus Houtt, the four supplementary medicinal herbs consisting of Glycyrrhiza uralensis Fischer, Mori cortex, Phaseoli radiati semen and red ginseng, which serve to increase the efficacies of the five main medicinal herbs, and horse fat rich in unsaturated fatty acids closer to vegetable oil components to animal oil components and in vitamin E, are optimally combined with one another by the herb combination methods for synergism and mutual enforcement according to the rules of seven emotions and combination used in Chinese medicine, they provide a natural medicinal herb extract for making a soap effective for atopic dermatitis patients.

Hereinafter, the effects of the method for making the herbal soap for alleviating atopic skin disease according to the preferred embodiment of the present invention will be described in detail.

According to the embodiment of the present invention, the five main medicinal herbs consisting of 1,000 g of Kalopanacis cortex, 4,000 g of Amaranthus mangostanus, 1,000 g of Gardenia jasminoides, 1,000 g of Lonicerae flos and 4,000 g of Rumex japonicus Houtt, the four supplementary medicinal herbs consisting of 1,000 g of Glycyrrhiza uralensis Fischer, 1,000 g of Mori cortex, 1,000 g of Phaseoli radiati semen and 500 g of red ginseng, which serve to increase the efficacies of the five main medicinal herbs, and 500 g of purified horse fat containing unsaturated fatty acids and vitamin E, are optimally combined with one another according to the above-described herb combination method used in Chinese medicine, and active ingredients showing alleviation effects effective for atopic dermatitis patients are extracted from the medicinal herbs. A soap composition containing this herbal extract and refined horse fat exhibits excellent effects on the fundamental treatment of diseases having a high correlation with atopic skin disease and on the alleviation of atopic skin disease.

As described above, the method for making the herbal soap for alleviating atopic skin disease according to the present invention has the following effects.

(1) The present invention provides a method for optimal combination of five main medicinal herbs, which alleviates atopic skin disease by fundamentally treating various skin diseases (particularly diseases such as stress, nephritis and hepatic diseases) having a direct connection and high correlation with atopic skin disease, and promote blood circulation and metabolic activity, four supplementary medicinal herbs serving to increase the efficacies of the five main medicinal herbs, and refined horse fat.

(2) The present invention provides a method for preparing a herbal soap composition for alleviating atopic skin disease, which contains components extracted from the medicinal herbs combined by the above combination method.

The foregoing is for illustrative purposes only, and those skilled in the art will appreciate that various modifications and variations are possible without departing from the essential characteristics of the present invention. Thus, the embodiments described above are considered to be illustrative in all respects and not restrictive, and the scope of the present invention is not limited by such embodiments. Furthermore, the scope of the present invention should be defined by the appended claims, and it should be understood that all modifications or variations derived from the meanings and scope of the present invention and equivalents thereof are included in the scope of the appended claims.

What is claimed is:

1. A method for making an herbal soap for alleviating atopic skin disease, the method comprising the steps of:
(1) preparing five main medicinal herbs consisting of Kalopanacis cortex, Amaranthus mangostanus, Gardenia jasminoides, Lonicerae flos and Rumex japonicus Houtt, and four supplementary medicinal herbs consisting of Glycyrrhiza uralensis Fischer, Mori cortex, Phaseoli radiati semen and red ginseng, which serve to increase the efficiencies of the five main medicinal herbs;
(2) introducing Amaranthus mangostanus, Rumex japonicus Houtt and Lonicerae flos, which have excellent effects on treatment and alleviation of atopic skin disease, among the five main medicinal herbs, into an herb medicine brewing pot together with purified water, and preheating the introduced herbs;
(3) after the preheating in step (2), stopping the heating, and aging the preheated herbs in a state in which an internal pressure of the herb medicine brewing pot was removed, thereby extracting a main raw material for preparing a soap composition;
(4) finely crushing the main medicinal herb Gardenia jasminoides and the supplementary medicinal herbs Glycyrrhiza uralensis Fischer, Mori cortex and Phaseoli radiati semen, prepared in step (1), finely cutting the red ginseng, introducing the crushed herbs and the cut red ginseng into the herb medicine brewing pot together with the main raw material extracted in step (3), and reheating the introduced herbs and main raw material;
(5) after the reheating in step (4), stopping the heating, and then aging the reheated herbs and main raw material in a state in which the internal pressure of the herb medicine brewing pot was removed;
(6) after the aging in step (5), reheating the content of the herb medicine brewing pot, followed by filtration, thereby recovering a raw material extract for making the soap composition; and
(7) transferring the raw material extract, obtained in step (6), to an agitator, and adding purified horse fat thereto, followed by gelling.

2. The method of claim 1, wherein the five main medicinal herbs prepared in step (1) are 1,000 g of Kalopanacis cortex, 4,000 g of Amaranthus mangostanus, 1,000 g of Gardenia jasminoides, 1,000 g of Lonicerae flos, and 4,000 g of Rumex japonicus Houtt.

3. The method of claim 1, wherein the four supplementary medicinal herbs prepared in step (1) are 1,000 g of Glycyrrhiza uralensis Fischer, 1,000 g of Mori cortex, 1,000 of Phaseoli radiati semen, and 500 g of red ginseng.

4. The method of claim 1, wherein the purified horse fat used in step (7) is added in an amount of 500 g.

5. The method of claim 1, wherein the preheating in step (2) is performed by introducing the medicinal herbs together with 15-20 L of purified water into a 20-30 L herb medicine brewing pot and preheating the introduced medicinal herbs under a pressure of 2-3 kgf/cm² at a temperature of 70° C. to 90° C. for 2-3 hours.

6. The method of claim 1, wherein the reheating in step (4) is performed in the herb medicine brewing pot under a pressure of 2-3 kgf/cm² at a temperature of 90° C. to 100° C. for 8-10 hours.

7. The method of claim 1, wherein the aging in step (3) and the aging in step (5) are each performed for 10-12 hours.

8. The method of claim 1, wherein the recovering of the raw material extract in step (6) is performed by reheating the content of the herb medicine brewing pot at a temperature of 100° C. to 120° C. for 2-3 hours and then filtering the medicinal herbs, contained in the herb medicine brewing pot, through a 200-mesh filter under a pressure of 2-3 kgf/cm².

9. The method of claim 1, wherein the gelling in step (7) is performed by heating the raw material extract and the horse fat at a temperature of 90° C. to 100° C. so as not to stick to the agitator, thereby evaporating water.
